(19) United States
(12) Patent Application Publication
(10) Pub. No.: US 2008/0172025 A1
(43) Pub. Date: Jul. 17, 2008

(54) CHEMICAL REACTION CARTRIDGE AND USING METHOD THEREOF

(75) Inventors: Takeo TANAAMI, Musashino-shi (JP); Atsushi ITO, Musashino-shi (JP); Hidetoshi AOKI, Musashino-shi (JP)

Correspondence Address: SUGHRUE MION, PLLC, 2100 PENNSYLVANIA AVENUE, N.W., SUITE 800, WASHINGTON, DC, 20037, US

(73) Assignee: YOKOGAWA ELECTRIC CORPORATION, Tokyo (JP)

(21) Appl. No.: 12/014,495

(22) Filed: Jan. 15, 2008

(30) Foreign Application Priority Data
Jan. 17, 2007 (JP) 2007-007807

(51) Int. Cl. G01N 33/48; A61B 19/00

(52) U.S. Cl. 604/414; 604/415; 422/68.1; 604/411

(57) ABSTRACT

Disclosed is a chemical reaction cartridge comprising: an elastic member as a component; a flow path and a plurality of chambers which are connected to each other by the flow path, the flow path or the chambers or both of the flow path and the chambers being partially blocked by applying external pressure to the elastic member from outside so that a fluid substance in the flow path or in the chambers is moved or blocked, and one of the chambers being a sample collecting chamber to temporarily retain a sample supplied from outside; and an injection needle to connect the sample collecting chamber and an outside portion of the sample collecting chamber.

### BACKGROUND OF THE INVENTION

1. Field of the Invention

The present invention relates to a chemical reaction cartridge and a using method thereof.

2. Description of Related Art

In Japanese Patent Application Laid Open Publication No. 2005-037368, for the purpose of especially performing a combination, a dissolution, a detection, a decomposition or the like of liquid solution in accordance with a predetermined protocol, without difference among individuals, in a low cost, safely and easily, a chemical reaction cartridge, comprising an elastic member and a substrate made of a rigid body, is suggested. In the elastic member, a chamber and a flow path are formed, and by the elastic member, it is possible that the fluid flows and the flow of the fluid is blocked in the chamber and in the flow path. The substrate is made of the rigid body so as to maintain the position and the shape of the cartridge.

According to the invention described in Japanese Patent Application Laid Open Publication No. 2005-037368, the elastic member is deformed by a roller or the like to flatten out the chamber and the flow path. By moving or stopping the roller or the like in the state of the chamber and the flow path being flattened out, the flow and the flow blocking of the fluid are performed.

The chemical reaction cartridge is also used as a cartridge for biology (as a biotip) for the purpose of examining and analyzing DNA, RNA, protein or the like. In the paragraph 0033 and FIG. 21 in Japanese Patent Application Laid Open Publication No. 2005-037368, an invention is disclosed in which a depressed portion is formed in an inlet of a sample such as blood, and the sample which has leaked is to be kept in the depressed portion. An injection needle is extracted from the cartridge.

In JP No. 3757412B, a biotip is disclosed, comprising a blood collecting tube which is inserted into an injection syringe to collect blood, and which is transparent with respect to an excitation light and an excited fluorescence; a rubber plug; a collecting section in which the blood that is collected is temporarily retained; a preprocessing section to perform a preprocessing such as a decomposition, an extraction, an addition of a fluorescent label, or the like; and a substrate in which a fragment of known DNA is disposed.

As described in the paragraph 0035-0038 in JP No. 3757412B, the collecting is completed by extracting the biotip from a blood collecting needle.

However, in the invention described in JP No. 3757412B, the blood collecting tube is used as a container for the biotip, and the blood is gradually injected to the deepest portion by using the inner pressure. Thus after the blood is collected, the blood cannot be timely transferred to the deepest portion by pressing a roller or the like, as in the invention described in Japanese Patent Application Laid Open Publication No. 2005-037368. Further, in the invention described in JP No. 3757412B, the blood collecting needle is extracted from the rubber plug of the biotip, and the blood may leak from the insert section of the rubber plug. Thus there may be danger of attaching the leaked blood to the surrounding substance.

According to the invention described in Japanese Patent Application Laid Open Publication No. 2005-037368, after the blood is collected, the blood can be timely transferred to the deepest portion or the like by pressing a roller or the like. According to the invention described in Japanese Patent Application Laid Open Publication No. 2005-037368, the injection needle is extracted from the cartridge. Because of the existence of the depressed portion surrounding an insert section, there is little danger of attaching the leaked sample to the surrounding substance. However, the danger of attaching the leaked sample to the surrounding substance may be caused. Further, the leaked sample is exposed to external air, and there may be danger depending on the material of the sample.

Moreover, apart from the cartridge, the blood collecting tube and the blood collecting needle or the like used for collecting blood have to be discarded, and therefore there is a problem that it is troublesome and dangerous.

### SUMMARY OF THE INVENTION

The present invention is made in view of the above described problems in the conventional technique, and a main object of the present invention is to provide a chemical reaction cartridge and the using method thereof, which can be handled safely when the sample is injected.

According to a first aspect of the present invention, there is provided a chemical reaction cartridge comprising:

an elastic member as a component;

a flow path and a plurality of chambers which are connected to each other by the flow path, the flow path or the chambers or both of the flow path and the chambers being partially blocked by applying external pressure to the elastic member from outside so that a fluid substance in the flow path or in the chambers is moved or blocked, and one of the chambers being a sample collecting chamber to temporarily retain a sample supplied from outside; and

an injection needle to connect the sample collecting chamber and an outside portion of the sample collecting chamber.

According to a second aspect of the present invention, there is provided a using method of the chemical reaction cartridge, comprising:

sticking the injection needle into an edge portion through which the injection needle is stuck, of the sample collecting container in which the sample is retained; and

pushing and injecting the sample into the sample collecting chamber through the injection needle by filling the sample collecting container with the fluid substance, in a state where an outer edge opening of the injection needle is disposed in the sample which is placed inside the sample collecting container.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1A is a plan view of a chemical reaction cartridge according to an embodiment of the present invention, FIG. 1B is a horizontal sectional view thereof, and FIG. 1C is a vertical sectional view thereof;

FIGS. 2A and 2B are horizontal sectional views of the chemical reaction cartridge and a blood collecting tube according to the embodiment of the present invention, and FIG. 2C is a vertical sectional view thereof;

FIG. 3A is a plan view showing a condition in which a sample is injected into the chemical reaction cartridge according to the embodiment of the present invention, and FIG. 3B is a plan view showing a condition in which another method is used;

FIG. 4 is a vertical sectional view of a chemical reaction cartridge according to another embodiment of the present invention; and

FIG. 5 is a vertical sectional view of a chemical reaction cartridge and a blood collecting tube or the like according to another embodiment of the present invention.

### DESCRIPTION OF THE PREFERRED EMBODIMENTS

Hereinbelow, embodiments of the present invention are described with reference to the drawings. In the description below, embodiments of the present invention are disclosed, and the present invention is not limited to these. Incidentally, the chemical reaction cartridge in the present invention is applied to a reactor vessel which is generally called as “a microreactor”. The present invention is not limited to a specific application.

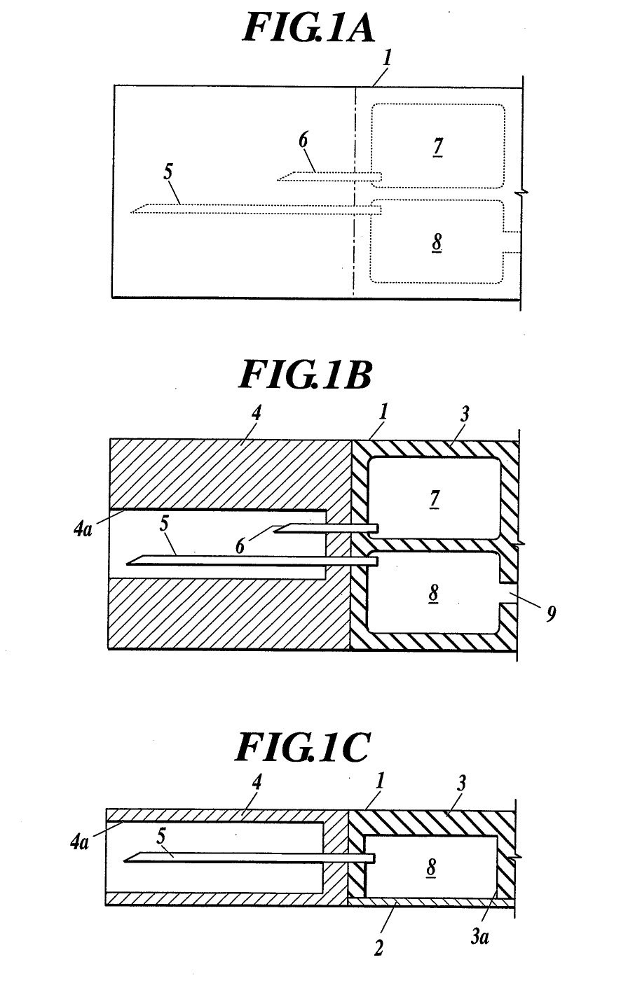

FIG. 1A is a plan view of a chemical reaction cartridge according to an embodiment of the present invention, FIG. 1B is a horizontal sectional view thereof, and FIG. 1C is a vertical sectional view thereof.

As shown in FIG. 1C, the chemical reaction cartridge 1 comprises an elastic member 3 such as rubber with airtightness and elasticity, and a substrate 2 made of a rigid body formed by a hard material for the purpose of determining a position and maintaining the shape of the chemical reaction cartridge. Further, a protecting member 4 is fixed on the edge portion of the chemical reaction cartridge 1.

As material of the elastic member 3, for example, silicone rubber, polydimethylsiloxane (PDMS), natural rubber and a polymer thereof, acrylic rubber, and polyurethane rubber or the like is used.

As material of the substrate 2, for example, glass, metal, hard resin, and a rigid body which can be bended or the like is used.

The protecting member 4 may be integrally formed with the substrate 2 by using the same material. Alternately, the protecting member 4 may be formed apart from the substrate 2 and be fixed to the substrate 2.

As shown in FIG. 1A, a depressed portion (for example, 3a) is formed on one surface of the elastic member 3. A portion of the surface of the elastic member 3, on which the depressed portion is formed, except the depressed portion, is attached to a surface of the substrate 2. Thus a flow path and chambers are formed. The number of the formed chambers is two or more. The flow path connects the chambers and makes a moving of a substance in between the chambers possible. The substance to be moved is for example, fluid substance, liquid and other substance with fluid property. When the reacting substance intended to be moved is one that does not flow or that is difficult to flow such as a solid substance, liquid solution including the reacting substance is placed into the chamber.

The flow path and the chambers may be formed so that they are entirely covered by the elastic member. Alternately, a portion of the wall section may comprise the elastic member. By positioning another layer of an elastic member in between the substrate 2 and the elastic member 3, the flow path and the chambers can be formed so that they are entirely covered by the elastic member. Alternately, an elastic member may be used instead of the substrate 2, and the cartridge may be comprised without the substrate made of the rigid body.

The moving of the substance is performed in a manner described below.

First, a pressing member such as a roller, a squeegee, a syringe or the like is pressed onto the flow path or the elastic member 3 on the chamber to flatten out the flow path or the chamber. By flattening out the flow path or the chamber, the substance inside can be flowed and can be moved. Further, by moving the pressing position, the substance inside can be flowed and can be moved in the moving direction of the pressing position. The moving of the pressing position is preferably performed in the following manner. That is, by contacting the internal walls opposing the flow path or the chamber at the pressing position, the flow path or the chamber is pressed to the state where the internal space is blocked.

The blocking of the moving substance is performed by contacting the internal walls opposing the flow path or the chamber and blocking the internal space by the pressing member. By using a plurality of pressing members, the following can be performed. That is, a substance may be moved by one pressing member. At the same time, the flow path or the chamber is pressed by another pressing member at the front of the substance in the moving direction to block the moving of the substance forward from the pressing position of another pressing member.

Based on the moving and the blocking described above, the moving of the substance in the cartridge 1 is controlled.

Based on the above described principle, the moving of the substance in the cartridge 1 is controlled, and an operation for a chemical reaction is performed. In the embodiment explained hereinbelow, the sample is blood, and the blood collecting tube is used as the sample collecting container.

As shown in FIG. 1B, in the cartridge 1, an ejection chamber 7, a sample collecting chamber 8, and a flow path 9 which is connected to the sample collecting chamber 8 are formed. The flow path 9 is connected to a preprocessing chamber (not shown).

In the sample collecting chamber 8, an injection needle 5 which penetrates the elastic member 3 and connects the sample collecting chamber 8 to an outside portion, is provided.

An ejection needle 6 is also provided parallel to the injection needle 5. The ejection chamber 7 is connected to an inner edge of the ejection needle 6. That is, the ejection needle 6 penetrates the elastic member 3 and connects the ejection chamber 7 to the outside portion.

The ejection chamber 7 is an independent chamber which is not connected to the sample collecting chamber 8 through the flow path and the chambers in the cartridge. The ejection chamber 7 itself may comprise a plurality of chambers connected by flow paths. Based on the above described principle, by pressing the elastic member 3 on the ejection chamber 7 by a roller or the like, air and other fluid substance inside the ejection chamber 7 can be ejected outside through the ejection needle 6.

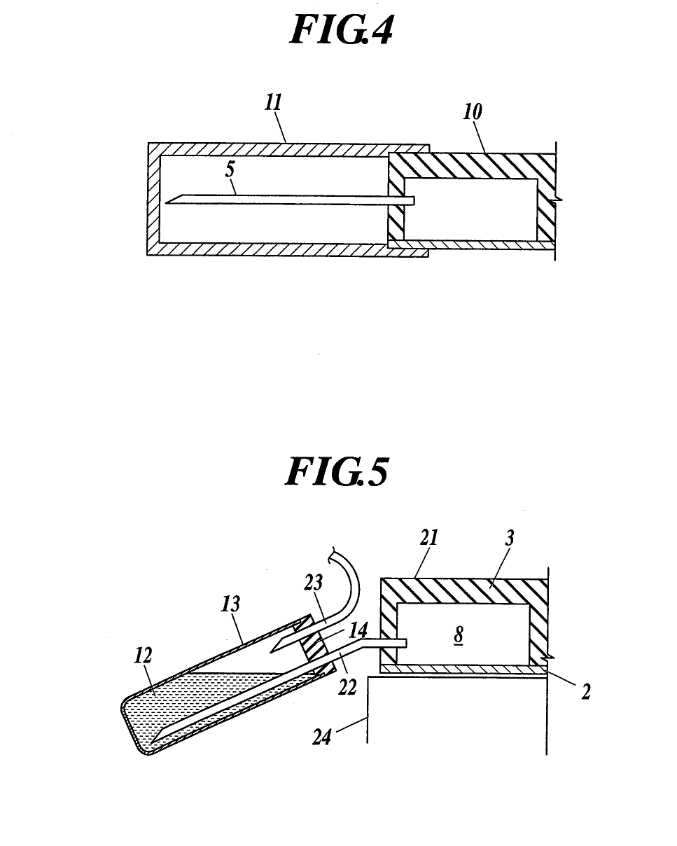

The injection needle 5 is longer than the ejection needle 6, and the outer edge portion of the injection needle 5 is further extended than that of the ejection needle 6. The injection needle 5 and the ejection needle 6 are surrounded by a hole section 4a of the protecting member 4. Incidentally, as shown in FIG. 4, instead of the protecting member 4, a cap member 11 which is removable from a cartridge main body 10 may be used as the protecting member of the needle.

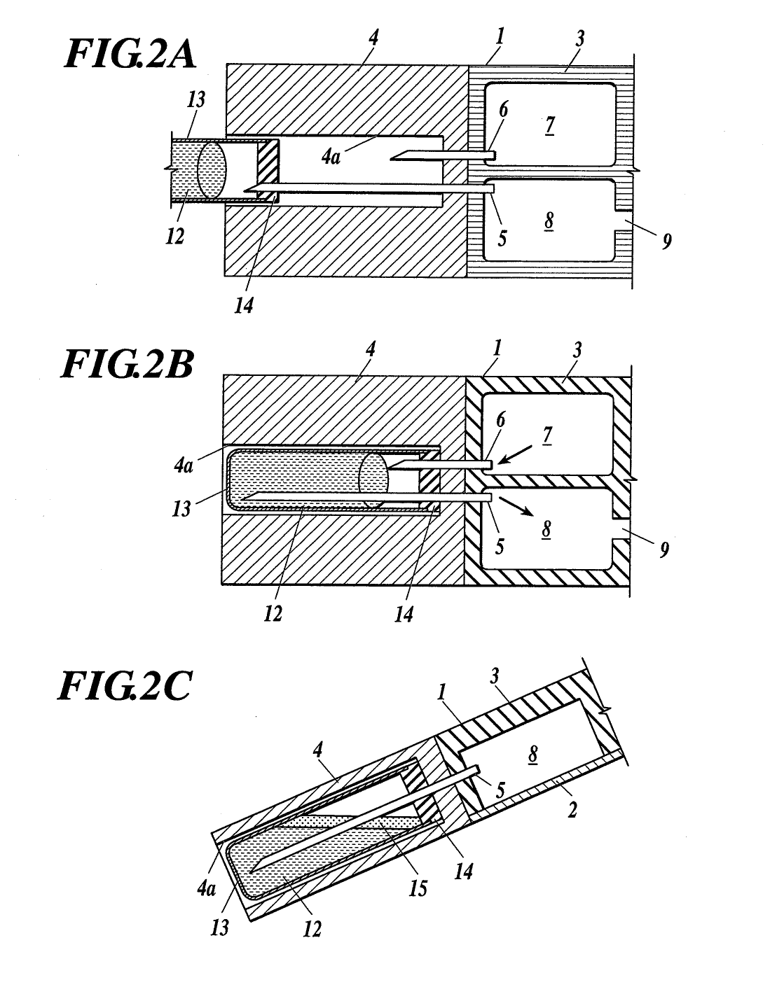

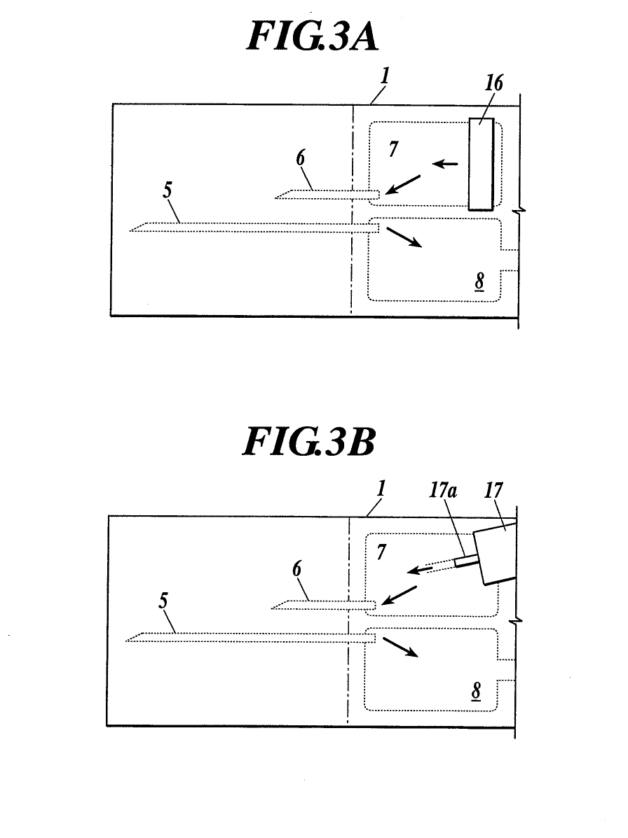

Next, the using method of the cartridge 1 is explained with reference to FIGS. 2A to 3B.

First, the blood collecting tube 13 in which blood 12 collected form a human body or the like by an injection syringe is retained, and the cartridge 1 are prepared. An opening edge of the blood collecting tube 13 is sealed by the rubber plug 14 through which the needles are stuck.

As shown in FIGS. 2A and 2B, the blood collecting tube 13 is inserted into the hole section 4a. At this point, the blood collecting tube 13 is inserted so as to direct an edge portion of the rubber plug 14 side forward in the inserting direction. While the blood collecting tube 13 is inserted into the hole section 4a in this manner, the injection needle 5 and the ejection needle 6 are stuck into the rubber plug 14. The blood collecting tube 13 is compressed to the farthest end of the hole section 4a, and the connection of the blood collecting tube 13 to the cartridge 1 is completed. The blood collecting tube 13 is held by the injection needle 5, the ejection needle 6, and the hole section 4a, to be integrated with the cartridge 1. In the state of being integrated in this manner, the injection of the blood from the blood collecting tube 13 to the sample collecting chamber 8, the examining of the blood, the discard of the blood collecting tube 13 and the cartridge 1 are performed.

Next, the injection of the blood from the blood collecting tube 13 into the sample collecting chamber 8 is performed.

First, as shown in FIGS. 2B and 2C, an outer edge opening of the injection needle 5 is disposed in the blood 12 inside the blood collecting tube 13. Further, an outer edge opening of the ejection needle 6 is disposed in a space outside the blood 12 inside the blood collecting tube 13. This state is suitably made by inclining the blood collecting tube 13 so that the rubber plug 14 side faces upward or the like, as shown in FIG. 2C.

In this state, the blood collecting tube 13 is filled with the air or the like from the ejection chamber 7 through the ejection needle 6. Thus, the blood 12 inside the blood collecting tube 13 is pushed out to be injected into the sample collecting chamber 8 through the injection needle 5.

The following two examples are disclosed as methods to fill the blood collecting tube 13 with the air or the like from the ejection chamber 7 through the ejection needle 6.

The first method is as follows. As shown in FIG. 3A, the elastic member on the ejection chamber 7 is pressed by the roller 16 to move the air to the air ejecting direction. Instead of the roller, another pressing member such as a squeegee, a syringe or the like may be used. In this method, it is necessary for the blood 12 to be pushed out by the air or the like inside the ejection chamber 7. Accordingly, it is necessary that the ejection chamber 7 have enough size to inject necessary amount of blood 12 into the sample collecting chamber 8.

The other method is as follows. As shown in FIG. 3B, an injection needle (a third needle) 17a of an injection syringe 17 is stuck into the ejection chamber 7 to send out the air or the like by the injection syringe 17. The blood collecting tube 13 is filled with the air or the like through the injection needle 17a, the ejection chamber 7 and the ejection needle 6. In this method, the air or the like to push out the blood 12 can be supplied from outside. Thus, it is only necessary that the ejection chamber 7 have enough size to easily be stuck through by the injection needle 17a.

The fluid substance with which the blood collecting tube 13 is filled through the ejection needle 6 is not limited to the air. The substance may be a fluid one which has no influence to the sample, such as oil and others. In order to perform the method shown in FIG. 3A, a cartridge in which the ejection chamber 7 is filled with a fluid substance which has no influence to the sample, such as air, oil and others, is used.

Alternatively, as shown in FIG. 2C, it is also effective to previously form an oil film 15 by filling the blood collecting tube 13 with oil, and subsequently filling up the blood collecting tube 13 with air. Thus mixing the air into the sample collecting chamber 8 is prevented.

As described above, the blood injection into the sample collecting chamber 8 is performed, and the injection needle 5 and the ejection needle 6 are stuck into the rubber plug 14. In the state where the blood collecting tube 13 and the cartridge 1 are connected to each other, the cartridge 1 is used for examining the blood. Subsequently, with keeping the same state, the blood collecting tube 13 and the cartridge 1 are discarded.

Next, a chemical reaction cartridge in another embodiment is shown with reference to FIG. 5.

A chemical reaction cartridge 21 shown in FIG. 5 does not comprise an ejection needle 23 and only comprises an injection needle 22. It is also preferable in this case that the injection needle 22 is provided with a protecting cap.

The injection needle 22 may be extended parallel to the substrate 2. However, the injection needle 22 in which the outer edge portion thereof is disposed so as to be inclined with respect to the substrate 2, is shown.

In a case where the cartridge 21 comprising the inclined injection needle 22 is used, only the blood collecting tube 13 can be inclined to a suitable angle for injecting the blood, in a state where the cartridge 21 is mounted on a horizontal table 24.

In the cartridge 21 shown in FIG. 5, because the ejection needle 23 is not attached, the ejection needle 23 is stuck into the rubber plug 14 at the time of the injection. The blood collecting tube is filled with the air or the like through the ejection needle 23 by an equipment such as a cylinder or the like (not shown).

In spite of the embodiments described above, a cartridge comprising an injection needle and an ejection needle, in which the both outer edge portions of the injection needle and of the ejection needle are inclined with respect to a substrate, may be applied. In this case, in a state where the cartridge is mounted horizontally, the pressing of the ejection chamber or the like can be performed by a roller or the like.

Alternatively, various modifications and combinations can be voluntarily applied such as a cartridge in which protecting members of an injection needle and of an ejection needle are fixed, or the protecting members thereof are provided so as to be removable, may be applied.

In the above described embodiment, blood is used as the sample. However, the sample which can be applied to the present invention is not limited to blood, and anything collected from a biological body, nature or the like may be used as the sample.

According to a first aspect of the preferred embodiments of the present invention, there is provided a chemical reaction cartridge comprising:

an elastic member as a component;

a flow path and a plurality of chambers which are connected to each other by the flow path, the flow path or the chambers or both of the flow path and the chambers being partially blocked by applying external pressure to the elastic member from outside so that a fluid substance in the flow path or in the chambers is moved or blocked, and one of the chambers being a sample collecting chamber to temporarily retain a sample supplied from outside; and

an injection needle to connect the sample collecting chamber and an outside portion of the sample collecting chamber.

Preferably, the chemical reaction cartridge comprises:

an ejection needle which is provided together with the injection needle; and

an ejection chamber which is connected to an inner edge of the ejection needle, and which ejects the fluid substance placed in the ejection chamber outside through the ejection needle by applying the external pressure to the elastic member from outside.

Preferably, the chemical reaction cartridge comprises:

an ejection needle which is provided together with the injection needle; and

an ejection chamber which is connected to an inner edge of the ejection needle, and in which at least a portion of a wall section is formed by the elastic member.

Preferably, the chemical reaction cartridge comprises a protecting member to surround the needle.

Preferably, the protecting member is a removable cap member.

Preferably, the chemical reaction cartridge comprises a protecting member to surround the injection needle and the ejection needle, wherein

the protecting member is fixed to a substrate.

Preferably, the protecting member comprises a hole section in which a sample collecting container having an edge portion through which the injection needle and the ejection needle are stuck is inserted, and which surrounds the injection needle and the ejection needle, and

the injection needle and the ejection needle are disposed so as to be stuck through the edge portion through which the injection needle and the ejection needle are stuck at the same time as an inserting operation in which the edge portion through which the injection needle and the ejection needle are stuck is inserted into the hole section.

Preferably, the ejection chamber is filled with the fluid substance which has no influence to the sample.

Preferably, the chemical reaction cartridge comprises the substrate made of a rigid body to maintain a form of a surface on which the flow path and the plurality of chambers are formed, wherein

an outer edge portion of the needle is disposed so as to be inclined with respect to the substrate.

According to a second aspect of the present invention, there is provided a using method of the chemical reaction cartridge, comprising:

sticking the injection needle into an edge portion through which the injection needle is stuck, of the sample collecting container in which the sample is retained; and

pushing and injecting the sample into the sample collecting chamber through the injection needle by filling the sample collecting container with the fluid substance, in a state where an outer edge opening of the injection needle is disposed in the sample which is placed inside the sample collecting container.

Preferably, a using method of the chemical reaction cartridge, comprises:

sticking the injection needle and the ejection needle into an edge portion through which the needles are stuck of the sample collecting container in which the sample is retained; and

pushing and injecting the sample into the sample collecting chamber through the injection needle by filling the sample collecting container with the fluid substance placed in the ejection chamber through the ejection needle by applying the external pressure to the elastic member from outside, in a state where an outer edge opening of the injection needle is disposed in the sample which is placed inside the sample collecting container and in a state where an outer edge opening of the ejection needle is disposed in a space where the sample in the sample collecting container is not placed.

Preferably, a using method of the chemical reaction cartridge, comprises:

sticking the injection needle and the ejection needle into an edge portion through which the needles are stuck, of the sample collecting container in which the sample is retained; and

pushing and injecting the sample into the sample collecting chamber through the injection needle by sticking a third needle to the elastic member and by filling the sample collecting container with the fluid substance from outside through the third needle, the ejection chamber and the ejection needle, in a state where an outer edge opening of the injection needle is disposed in the sample which is placed in the sample collecting container and in a state where an outer edge opening of the ejection needle is disposed in a space where the sample in the sample collecting container is not placed.

Preferably, a using method of the chemical reaction cartridge, comprises:

connecting the sample collecting container and the chemical reaction cartridge to each other by inserting the edge portion through which the needles are stuck, of the sample collecting container in which the sample is retained, into the hole section of the protecting member, and by sticking the injection needle and the ejection needle into the edge portion.

Preferably, the chemical reaction cartridge is used for examining the sample in a state where at least the injection needle is stuck into the edge portion through which at least the injection needle is stuck, of the sample collecting container.

Preferably, the chemical reaction cartridge is used for examining the sample, and the sample collecting container and the chemical reaction cartridge are discarded, in the state where at least the injection needle is stuck into the edge portion through which at least the injection needle is stuck, of the sample collecting container.

Preferably, the sample is injected from the sample collecting container into the sample collecting chamber in a state where the needle or the chemical reaction cartridge is inclined with respect to a direction of a gravitational force.

According to the embodiments of the present invention, the sample collecting container and the chemical reaction cartridge are connected to each other, are used integrally, and are discarded. Thus, the leaking of a minute sample is prevented, the sample is safely injected to the chemical reaction cartridge, and used instruments including the chemical reaction cartridge can be discarded safely.

The entire disclosure of Japanese Patent Application No. 2007-007807 filed on Jan. 17, 2007 including description, claims, drawings, and abstract are incorporated herein by reference in its entirety.

Although various exemplary embodiments have been shown and described, the invention is not limited to the embodiments shown. Therefore, the scope of the invention is intended to be limited solely by the scope of the claims that follow.

What is claimed is:

1. A chemical reaction cartridge comprising:
an elastic member as a component;
a flow path and a plurality of chambers which are connected to each other by the flow path, the flow path or the chambers or both of the flow path and the chambers being partially blocked by applying external pressure to the elastic member from outside so that a fluid substance in the flow path or in the chambers is moved or blocked, and one of the chambers being a sample collecting chamber to temporarily retain a sample supplied from outside; and
an injection needle to connect the sample collecting chamber and an outside portion of the sample collecting chamber.

2. The chemical reaction cartridge of claim 1, comprising:
an ejection needle which is provided together with the injection needle; and
an ejection chamber which is connected to an inner edge of the ejection needle, and which ejects the fluid substance placed in the ejection chamber outside through the ejection needle by applying the external pressure to the elastic member from outside.

3. The chemical reaction cartridge of claim 1, comprising:
an ejection needle which is provided together with the injection needle; and
an ejection chamber which is connected to an inner edge of the ejection needle, and in which at least a portion of a wall section is formed by the elastic member.

4. The chemical reaction cartridge of claim 1, comprising a protecting member to surround the needle.

5. The chemical reaction cartridge of claim 4, wherein the protecting member is a removable cap member.

6. The chemical reaction cartridge of claim 2, comprising a protecting member to surround the injection needle and the ejection needle, wherein
the protecting member is fixed to a substrate.

7. The chemical reaction cartridge of claim 6, wherein
the protecting member comprises a hole section in which a sample collecting container having an edge portion through which the needle is stuck is inserted, and which surrounds the needle, and
the needle is disposed so as to be stuck through the edge portion through which the needle is stuck at the same time as an inserting operation in which the edge portion through which the needle is stuck is inserted into the hole section.

8. The chemical reaction cartridge of claim 2, wherein the ejection chamber is filled with the fluid substance which has no influence to the sample.

9. The chemical reaction cartridge of claim 1, comprising the substrate made of a rigid body to maintain a form of a surface on which the flow path and the plurality of chambers are formed, wherein
an outer edge portion of the needle is disposed so as to be inclined with respect to the substrate.

10. A using method of the chemical reaction cartridge of claim 1, comprising:
sticking the injection needle into an edge portion through which the injection needle is stuck, of the sample collecting container in which the sample is retained; and
pushing and injecting the sample into the sample collecting chamber through the injection needle by filling the sample collecting container with the fluid substance, in a state where an outer edge opening of the injection needle is disposed in the sample which is placed inside the sample collecting container.

11. A using method of the chemical reaction cartridge of claim 2, comprising:
sticking the injection needle and the ejection needle into an edge portion through which the needles are stuck of the sample collecting container in which the sample is retained; and
pushing and injecting the sample into the sample collecting chamber through the injection needle by filling the sample collecting container with the fluid substance placed in the ejection chamber through the ejection needle by applying the external pressure to the elastic member from outside, in a state where an outer edge opening of the injection needle is disposed in the sample which is placed inside the sample collecting container and in a state where an outer edge opening of the ejection needle is disposed in a space where the sample in the sample collecting container is not placed.

12. A using method of the chemical reaction cartridge of claim 3, comprising:
sticking the injection needle and the ejection needle into an edge portion through which the needles are stuck, of the sample collecting container in which the sample is retained; and
pushing and injecting the sample into the sample collecting chamber through the injection needle by sticking a third needle to the elastic member and by filling the sample collecting container with the fluid substance from outside through the third needle, the ejection chamber and the ejection needle, in a state where an outer edge opening of the injection needle is disposed in the sample which is placed in the sample collecting container and in a state where an outer edge opening of the ejection needle is disposed in a space where the sample in the sample collecting container is not placed.

13. A using method of the chemical reaction cartridge of claim 7, comprising:
connecting the sample collecting container and the chemical reaction cartridge to each other by inserting the edge portion through which the needles are stuck, of the sample collecting container in which the sample is retained, into the hole section of the protecting member, and by sticking the injection needle and the ejection needle into the edge portion.

14. The using method of the chemical reaction cartridge of claim 10, wherein
the chemical reaction cartridge is used for examining the sample in a state where the needle is stuck into the edge portion through which the needle is stuck, of the sample collecting container.

15. The using method of the chemical reaction cartridge of claim 14, wherein
the chemical reaction cartridge is used for examining the sample, and the sample collecting container and the chemical reaction cartridge are discarded, in the state where the needle is stuck into the edge portion through which the needle is stuck, of the sample collecting container.

16. The using method of the chemical reaction cartridge of claim 10, wherein
the sample is injected from the sample collecting container into the sample collecting chamber in a state where the needle or the chemical reaction cartridge is inclined with respect to a direction of a gravitational force.
